(19) United States
(12) Patent Application Publication
(10) Pub. No.: US 2015/0141866 A1
(43) Pub. Date: May 21, 2015

(54) SYSTEM AND METHOD FOR EVALUATION OF THE PLEURAL SPACE

(71) Applicants: Martin L. Mayse, Wayzata, MN (US); Steven C. Dimmer, Bellevue, WA (US)

(72) Inventors: Martin L. Mayse, Wayzata, MN (US); Steven C. Dimmer, Bellevue, WA (US)

(21) Appl. No.: 14/543,076

(22) Filed: Nov. 17, 2014

Related U.S. Application Data
(60) Provisional application No. 61/905,560, filed on Nov. 18, 2013.

(51) Int. Cl. A61B 10/04; A61B 1/267

(52) U.S. Cl. CPC A61B 10/04; A61B 1/267; A61B 2010/009
USPC 600/564; 600/562

(57) ABSTRACT

Method and device are provided for evaluating the pleural space. The device in introduced percutaneously into a pleural space. The device includes an energy detector that facilitates evaluation of the pleura. The device also includes a tissue collection component and a joint. The joint allows the device to be bent at least 120 degrees, facilitating assessment of the pleura. The device allows tissue sampling when the device is bent.

The method encompasses selective tissue sampling of the parietal pleura without inducing pneumothorax, with its attendant extended recovery time, in the patient. The distal portion of the device is introduced percutaneously into the pleural space. The pleura near the access point is evaluated using an energy emitter and energy receiver integrated into the device and a portion of the parietal pleura near the access point is sampled using a biopsy tool integrated into the distal portion of the device.

### CROSS-REFERENCES TO RELATED APPLICATIONS

This application claims the benefit of U.S. Provisional Application No. 61/905,560, filed Nov. 18, 2013, the entire contents and substance of which are hereby incorporated in total by reference.

### CITED REFERENCES

The Light RW: Approach to the patient. Pleural Diseases, 4th Ed., Lippincott Williams & Wilkins, Philadelphia, 2001, p 87.
Alemán C, Alegre J, Armadans L, et al: The value of chest roentgenography in the diagnosis of pneumothorax after thoracentesis. Am J Med 107:340, 1999.
Bartter T, Mayo P D, Pratter M R: Lower risk and higher yield for thoracentesis when performed by experienced operators. Chest 103:1873, 1993.
Light R W, Macgregor M I, Luchsinger P C, et al: Pleural effusions: the diagnostic separation of transudates and exudates. Ann Intern Med 77:507, 1972.
McVay P A, Toy P T: Lack of increased bleeding after paracentesis and thoracentesis in patients with mild coagulation abnormalities. Transfusion 31:164, 1991.
Loddenkemper R: Thoracoscopy: state of the art. Eur Respir J 11:213, 1998.
Pastis N J, Nietert P J, Silvestri G A. et al: Variation in training for interventional pulmonary procedures among US pulmonary/critical care fellowships. Chest 127:1614, 2005.
Kirsch C M, Kroe D M, Azzi R L, et al: The optimal number of pleural biopsy specimens for a diagnosis of tuberculous pleurisy. Chest 112:702, 1997.

### BACKGROUND

The pleura is the serous membrane that covers the lung parenchyma, the mediastinum, the diaphragm, and the rib cage. The visceral pleura covers the outer surface of the lung parenchyma; including the interlobar fissures. The parietal pleura lines the inside of the thoracic cavity. Normally pleural fluid consists of a thin film between the visceral and parietal pleura, where it acts as a lubricant and mechanically couples the lung to chest wall. This fluid space is normally thin.

Under normal conditions, the rate of pleural fluid formation and absorption are in dynamic balance and the amount of fluid present in the pleural space is relatively constant. However, under certain abnormal conditions, such as infection, inflammation, malignancy, heart failure, liver failure, or kidney failure, among other conditions, the rates of pleural fluid formation and absorption within the pleural cavity becomes unbalanced resulting in the resulting in a net accumulation of fluid in the pleural space. This accumulation of pleural fluid is known as pleural effusion.

The presence of a pleural effusion is indicative of an underlying pathology. Because of this, the cause of the effusion is investigated in order to uncover and treat the underlying pathology.

It has been estimated that almost 1.4 million people develop pleural effusion each year in the United States. A wide variety of pathologies can lead to the development of a pleural effusion, but ninety percent of effusions are caused by five processes: congestive heart failure (36%), pneumonia (22%), malignancy (14%), pulmonary embolism (11%) and viral infections (7%). In general, pleural effusion resulting from congestive heart failure can be diagnosed without invasive diagnostic testing. However, other causes generally require invasive diagnostic testing to determine the underlying pathology.

Thoracentesis is often the first invasive diagnostic test performed in the evaluation of a pleural effusion of unknown etiology. It involves the aspiration of fluid from the pleural space. Imaging studies demonstrating free flowing fluid in the pleural space (e.g., decubitus chest radiographs, chest ultrasonography, or computed tomographs (“CT”) of the chest) are reviewed prior to thoracentesis. The patient is typically placed in an upright, seated position with their back as vertical to the floor as possible and with the arms folded and resting on a bedside table. The proper site for insertion of the needle is in the mid to lateral two thirds of the posterior hemithorax, avoiding the paraspinal region, and in the rib interspace approximately 1 cm inferior to the level where the percussion note becomes dull. Different sites may be chosen as directed by imaging guidance, such as ultrasound. The site should be prepped and draped using standard sterile technique. Topical anesthesia is provided to the selected area, with special attention paid to the skin surface, the superior margin of the rib, and the parietal pleura. The thoracentesis needle, typically a 1.5 inch long 22-gauge needle (˜0.72 mm OD), is then passed though the anesthetized area and into the pleural space. The needle is held in place so that the tip does not move relative to the patient's chest wall. Fluid can then be aspirated. Various commercially available needle-catheter systems are available for thoracentesis and are used for both diagnostic and therapeutic purposes.

Post-thoracentesis chest radiographs are often helpful to quantify the result of the thoracentesis and to evaluate those portions of the chest previously obscured by effusion fluid. Generally, post-thoracentesis chest radiographs are not necessary to rule-out pneumothorax, unless the patient develops symptoms during the procedure. In one report of 506 patients who underwent both thoracentesis and post-procedure chest radiographs, only one patient (0.2%) developed a large pneumothorax without associated symptoms. Complications of thoracentesis when performed by experienced operators without image guidance include pain (26%), cough (24%), worsening dyspnea (8%), pneumothorax (4%), dry tap (2%), and vagal reaction (2%). Complication rates are higher when thoracentesis is performed by inexperienced operators. Ultrasound guidance during thoracentesis can decrease the rate of complications, especially for pneumothorax (2.5%) and dry tap (0.3%).

Analysis of pleural effusion fluid begins by simple inspection (i.e., noting color, smell, and turbidity) and is followed by chemical analysis, which allows categorization of the effusion as transudative or exudative. Transudative pleural effusions occur when alterations of hydrostatic and oncotic factors increase the formation or decrease the absorption of pleural fluid; as may occur in congestive heart failure, cirrhosis, or nephrotic syndrome. Exudative pleural effusions occur when damage to, or disruption of, the normal pleural membranes or vasculature leads to increased capillary permeability or decreased lymphatic drainage; as may occur with infection, tumor involvement of the pleural space, and other inflammatory conditions. Pleural fluid is categorized as an exudate if it has any of the following criteria: (1) a pleural fluid-to-serum protein ratio of greater than 0.5, (2) a pleural fluid-to-serum LDH ratio of greater than 0.6, or (3) a pleural fluid LDH of more than two thirds of the upper limit of normal for serum LDH. Effusions that meet none of these three criteria are categorized as transudates. Although categorization of pleural fluid as transudative or exudative can be useful, the results of such analysis must be interpreted in the clinical context.

Thoracentesis should be performed in patients with a pleural effusion of unknown etiology. Thoracentesis is also indicated in patients with long-standing pleural effusion (1) when the patient develops a fever without a clear cause, (2) when an air-fluid level develops within the pleural space, (3) when there is a rapid change in the size of the effusion, or (4) when empyema may be developing. Thoracentesis should not be attempted in uncooperative patients and or patients who have an uncorrectable bleeding diathesis (e.g., prothrombin time or partial thromboplastin time greater than two times normal, a platelet count less than 50,000/mm, or a creatinine level greater than 6 mg/dl).

On the basis of pleural fluid analysis, the sensitivity of thoracentesis is 62% for the diagnosis of malignancy and 28% for tuberculosis. Unfortunately, a specific diagnosis can be made on the basis of pleural fluid analysis alone the minority of cases.

Pleural effusions for the following conditions may be definitively diagnosed based on pleural fluid analysis: Malignancy by cytology positive; Empyema by pus or stain or culture positive; Tuberculous pleurisy by stain or culture positive; Fungal pleurisy by stain or culture positive; Hemothorax by hematocrit ratio, pleural to blood greater than 0.5; Lupus pleuritis by cytology showing LE cells, ANA ratio, pleural to serum greater than 1.0; Rheumatoid pleurisy by cytology showing characteristic cells; Chylothorax by chylomicrons present, triglycerides greater than 110 mg per dl; Esophageal rupture by salivary amylase present; and Urinothorax by creatinine ratio, pleural to serum greater than 1.0. As noted above, in the majority of cases, pleural fluid analysis does not provide a definitive diagnosis. In such cases, a number of tissue sampling methods are used to diagnose the cause of the observed pleural effusion.

Closed pleural biopsy is an adjunct invasive diagnostic procedure in which a tissue specimen is sampled blindly (e.g. without any direct visualization) from the parietal pleura. It is performed using a large-bore needle with a cutting edge capable of removing tissue samples from the parietal pleura. Site selection is similar to that for thoracentesis. The presence of pleural fluid at the site of biopsy will decrease the risk of injury to the lung, and site selection should take this into consideration. Biopsies are collected from the superior edge of the rib located below the needle in order to avoid the neurovascular bundle that resides along the inferior margin of the rib. FIG. 1 shows an Abrahm's needle that has been passed through the chest wall and is in position to collect a tissue specimen from the parietal pleura.

In decades past, closed pleural biopsy was a standard approach in the diagnostic evaluation of pleural pathology; now it is almost a lost skill. With the exception of tuberculous pleural effusion, closed pleural biopsy has no significant incremental diagnostic value over thoracentesis; as such, its only indication is when tuberculous pleural effusion is suspected. When closed pleural biopsy is performed, a minimum of six samples should be collected and sent for culture and histology; the resultant sensitivity for tuberculous pleural effusion is 87%.

Transthoracic needle aspiration and biopsy is an adjunct to thoracentesis and can be useful in the evaluation of pleural disease when abnormalities such as pleural thickening, pleural nodularity, pleural masses or chest wall masses are demonstrated on thoracic imaging. Passage of the needle can be guided by ultrasound or CT. Contraindications and risks are the same as those for thoracentesis listed above. Complications are quite rare, but bleeding, pneumothorax, effusion, and infection can occur. When a specific lesion is visualized, the sensitivity for malignancy is over 85% and specificity is high.

Thoracoscopy is an invasive diagnostic procedure in which a tissue specimen is sampled under direct visualization from the parietal pleura. The patient is placed in a lateral decubitus position. The patient is turned to rest on one side of his or her trunk, with the dependent (down) side naming the position. Right lateral decubitus is right side down for a left sided procedure. Left lateral decubitus is left side down for a right sided procedure. The patient can be given conscious sedation with local anesthesia and allowed to breathe spontaneously, or the patient can be intubated with either a single or double lumen endotracheal tube and given general anesthesia. A single intercostal (between the ribs) incision is commonly used to insert a rigid optical forceps or a videoscope. Pleural fluid is evacuated and air is allowed to enter enabling unobstructed and direct visualization of the pleural surfaces. A detailed inspection of the pleural space is then undertaken and biopsy specimens are taken from any identified region of abnormality, using either rigid optical forceps or flexible biopsy forceps that are passed through the working channel of a videoscope. Following the procedure a drainage tube is left in the chest to evacuate the air that was introduced during drainage of the effusion fluid. The chest drainage tube is left in place and the patient kept in the hospital for 1-7 days following the thoracoscopy procedure before removal of the tube and discharge from the hospital.

Thoracoscopy is most commonly used diagnostically in the evaluation of pleural effusions following one or two non-determinative thoracenteses. Specific contraindications for thoracoscopy include extensive pleural adhesions, the patient's inability to lay in a lateral decubitus position, and the patient's inability to tolerate an induced pneumothorax.

The current state of the art in flexible pleuroscopes used in thoracoscopy is the Olympus LTF-160. This scope has a 7.0 mm outer diameter, 2.8 mm working channel (through which biopsy devices are inserted) and a 270 mm working length. Limitations of this scope with regards to evaluation the pleural space include: requires evacuation of pleural space fluids before usage—the user may not be able to view the parietal or visceral pleural surfaces without first evacuating the pleural space of fluid by inducing a pneumothorax; small biopsy sample size—biopsy devices (e.g. forceps) are limited in size due to the scope working diameter, this results in relatively small biopsy sample sizes which necessitates the need for additional biopsy samples and/or less than optimal diagnosis due to limited tissue available for pathology evaluation; requires relatively large incision—the scope has an outer diameter of 7 mm (cross sectional area of approximately 38.5 mm2) and may be used with an 8 mm trocar (cross sectional area of approximately 50.3 mm2); tissue grasping can be difficult and taking a biopsy of the pleura can be difficult, due to the resistance of the tissue and the long scope working length, pleural tissue is harder than, for example, bronchial tissue and not easy to grasp; risk of infection/inconvenience with reusable scope—reusable medical devices increase the risk of infection and cross contamination (relative to a single-use device) due to the potential for a inadequately sterilized device to be used with a patient's procedure, also, since the scope must be sterilized between pleural disease procedures the facility must either have multiple scopes available or wait for the scope to be sterilized between procedures.

For pleural malignancy, thoracoscopy has a diagnostic sensitivity of 95%. False negatives may result from insufficient biopsies or pleural adhesions that limit access to the involved areas of the pleura. The yield of thoracoscopy for malignant involvement of the pleura does not vary between lung carcinomas, mesotheliomas, or primary extrathoracic cancers that are metastatic to the chest. For tuberculous pleural effusions, thoracoscopy has a diagnostic sensitivity of 94% on the basis of histology alone and 99% when histology is combined with mycobacterial culture. Mycobacterial cultures are twice as likely to be positive from specimens obtained by thoracoscopy than those obtained by thoracentesis or closed pleural biopsy. The pathologic finding of pleuritis is nonspecific and can occur in a wide variety of disease states including nontuberculous pleural infection, connective tissue diseases, post-myocardial injury syndromes, and, occasionally, pulmonary embolism.

Accordingly, it is desirable to increase diagnostic sensitivity and accuracy and reduce the pain, complications, and length of hospitalization experienced by patients. Furthermore, other desirable features and characteristics of the present invention will become apparent from the subsequent detailed description of the invention and the appended claims, taken in conjunction with the accompanying drawings and this background of the invention.

### BRIEF DESCRIPTION OF THE DRAWINGS

The present invention will hereinafter be described in conjunction with the following drawing figures, wherein like numerals denote like but not necessarily identical elements.

FIG. 1 (Prior art) is a cross section of the chest wall with an Abrahm's needle in place collecting a tissue specimen from the parietal pleural.

FIG. 2 (Prior art) is an illustration of conventional method of direct visual inspection of the pleural space using a thoracoscope.

FIG. 3 is an illustration of the new system and method of direct visual inspection of the pleural space.

FIG. 4 is an elevational view of the new device with the tissue collection component open and the elongate member in the straight position.

FIG. 5 is an elevational view of the new device with the tissue collection component closed and the elongate member in the straight position.

FIG. 6 is an elevational view of the tissue collection component located at the distal end of the elongate member in the open position demonstrating a fluid transmission component.

FIG. 7 is an elevational view of the tissue collection component located at the distal end of the elongate member in the closed position.

FIG. 8 is an elevational view of the new device with the tissue collection component open and the elongate member flexed.

FIG. 9 is an elevational view of the new device with the tissue collection component closed and the elongate member flexed.

FIG. 10 is a cross sectional view of the chest wall with the elongate member of the new device passed through the chest wall with the elongate member flexed to a first position to bring the tissue collection component into close proximity to a first portion of the parietal pleural.

FIG. 11 is a cross sectional view of the chest wall with the elongate member of the new device passed through the chest wall with the elongate member flexed to a second position to bring the tissue collection component into close proximity to a second portion of the parietal pleural.

FIG. 12 is an elevational view of an alternative embodiment of the distal end of the elongate member of the device with the tissue collection component in the open position with a fluid transmission component located beside the tissue collection component.

FIG. 13 is an elevational view of the alternative embodiment in FIG. 12 shown with the tissue collection component in the closed position.

FIG. 14 is an elevational view of an alternative embodiment of the tissue collection component located at the distal end of the elongate member in the open position.

FIG. 15 is an elevational view of an alternative embodiment of the tissue collection component located at the distal end of the elongate member with fluid containment extensions in the open position.

FIG. 16 is an elevational view of an alternative embodiment of the tissue collection component located at the distal end of the elongate member with fluid containment extensions in the closed position.

FIG. 17 is an elevational view of an alternative embodiment of the tissue collection component located at the distal end of the elongate member in the open position with the tissue collection component constructed of transparent material and the energy emitter, energy detector, and fluid transmission component enclosed within the tissue collection component.

FIG. 18 is a view of the chest wall imaging plane as seen from the inside of the patient's body.

FIG. 19 is a view of the flexion of the elongate member adjusted at different angles to define a circular search pattern.

FIG. 20 is a view of an embodiment which has two locations whereby the elongate member flexes which enables an expanded circular search pattern with a single location of implantation.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

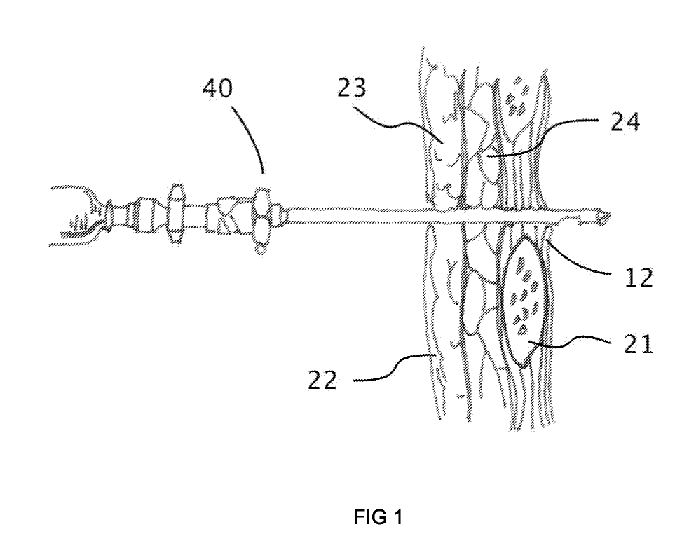

FIG. 1 shows a cross section of the chest wall with an Abrahm's needle (40) that has been passed through the skin (22), fat (23), and muscle (24) of the chest wall (20) along the superior margin of a rib (21) into a pleural effusion (15) and is in position to collect a tissue specimen from the parietal pleura (12). The tissue specimen is sampled blindly (i.e. without any direct visualization) from the parietal pleura (12) along the superior edge of a rib (21) in order to avoid the neurovascular bundle that resides along the inferior margin of the rib. The lack of direct tissue visualization limits the ability to reliably obtain a tissue specimen from a diseased area of the parietal pleura (12) and with the exception of tuberculous pleural effusion this method has no significant incremental diagnostic value over thoracentesis.

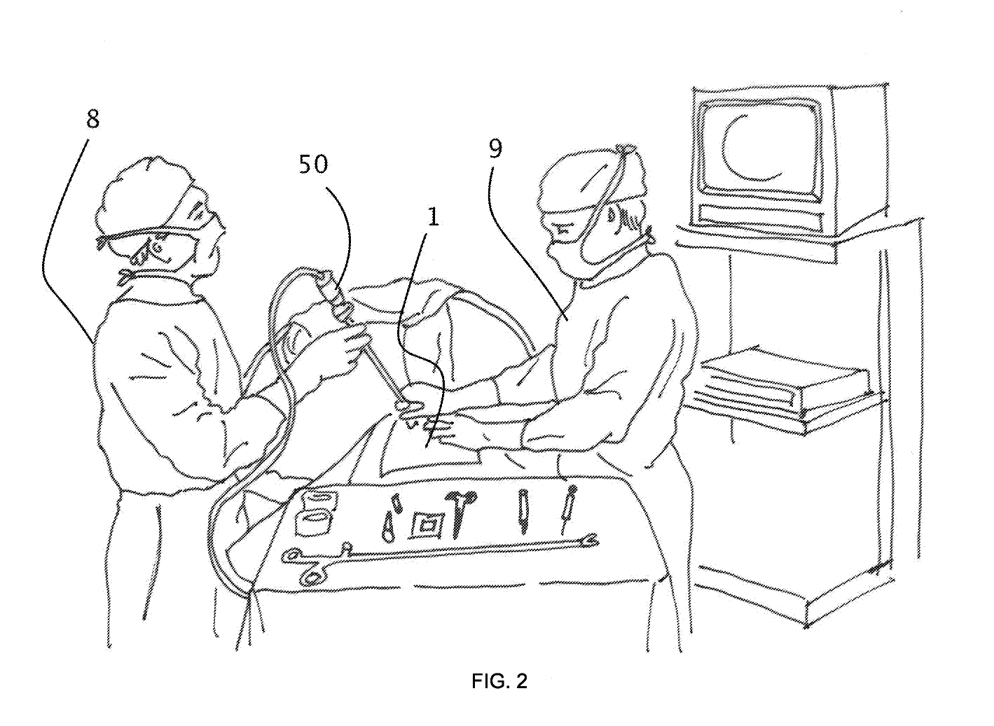

FIG. 2 shows the conventional method of direct visual inspection of the pleural space (10) using a standard thoracoscope (50). The patient (1) is placed on their side in a lateral decubitus position. The patient (1) can be given conscious sedation with local anesthesia and allowed to breathe spontaneously or the patient can be intubated with either a single or double lumen endotracheal tube and given general anesthesia. An operator (8) and an assistant (9) stand on either side of the patient (1). A single intercostal incision is commonly used to insert a thoracoscope (50). Pleural fluid (15) is evacuated and air is allowed to enter the pleural space enabling direct visualization of the visceral (13) and parietal (12) pleural surfaces. An inspection of the pleural space (10) is then undertaken and biopsy specimens are taken from any identified region of abnormality. Following the procedure a drainage tube is left in the chest to evacuate the air that was introduced during drainage of the effusion fluid (15). The chest drainage tube is left in place and the patient kept in the hospital for 1-7 days following the procedure before removal of the tube and discharge from the hospital.

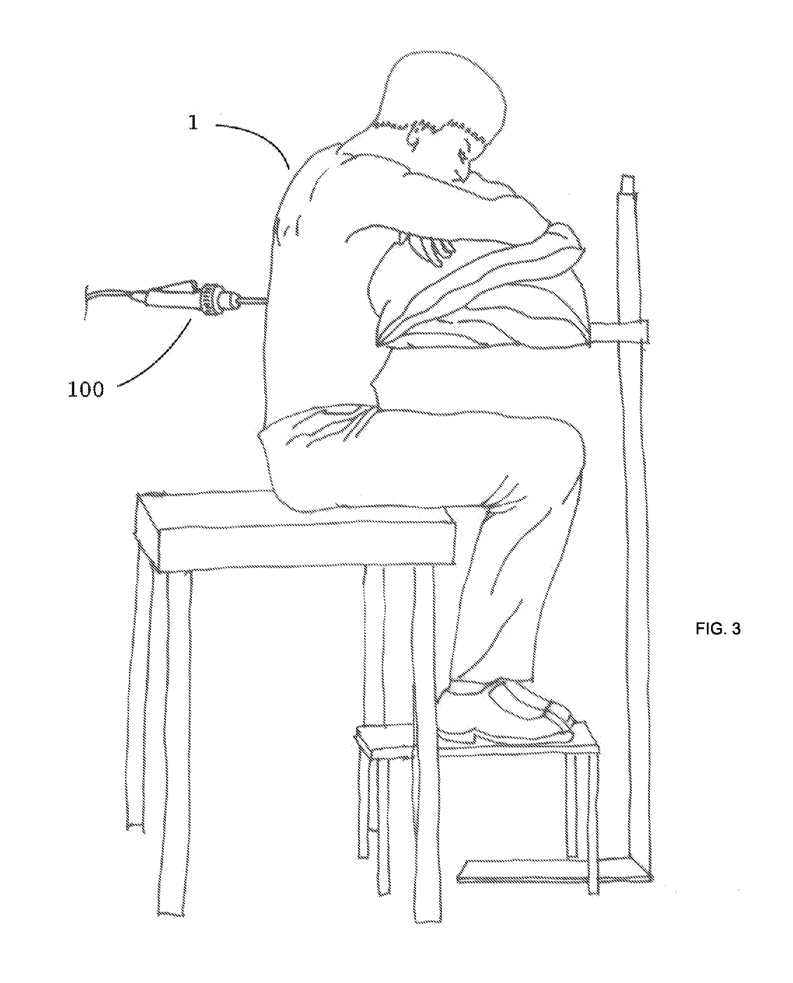

FIG. 3 is an illustration of the new method of direct visual inspection of the parietal pleural (12) surface described herein. The patient (1) is placed in an upright, seated position with their back as vertical to the floor as possible and with the arms folded and resting on a bedside table. The first step is selection of the insertion site in the mid to lateral two thirds of the posterior hemithorax, avoiding the paraspinal region, and between adjacent ribs just superior to the inferior rib (21) approximately 1 cm inferior to the level of the pleural effusion. This can be determined by physical exam or by imaging guidance, such as ultrasound. The site should be prepped and draped using standard sterile technique. Topical anesthesia is provided through a needle (not shown) to the selected area, with special attention paid to the skin (22) surface, the superior margin of the rib (21), and the parietal pleura (12) itself. A small skin incision is then made in the skin (22) of the chest wall (20) and the elongate member (110) of the scope (100) is passed though the incision and into the pleural space (10). A console (not shown) that provides a display, a power source, and/or a fluid. In one embodiment, the console includes a clear fluid such as normal saline and a pump which is used to infuse the fluid in a controlled manner near the energy detector (112) to displace turbid pleural fluid and improve visibility. Since this new procedure does not induce a pneumothorax (and does not require a chest tube) the patient may be able to return home on the day of the procedure. This new procedure reduces or eliminates the need for a patient to stay overnight in the hospital to recover from the diagnostic procedure. The new procedure reduces patient morbidity and procedure related costs.

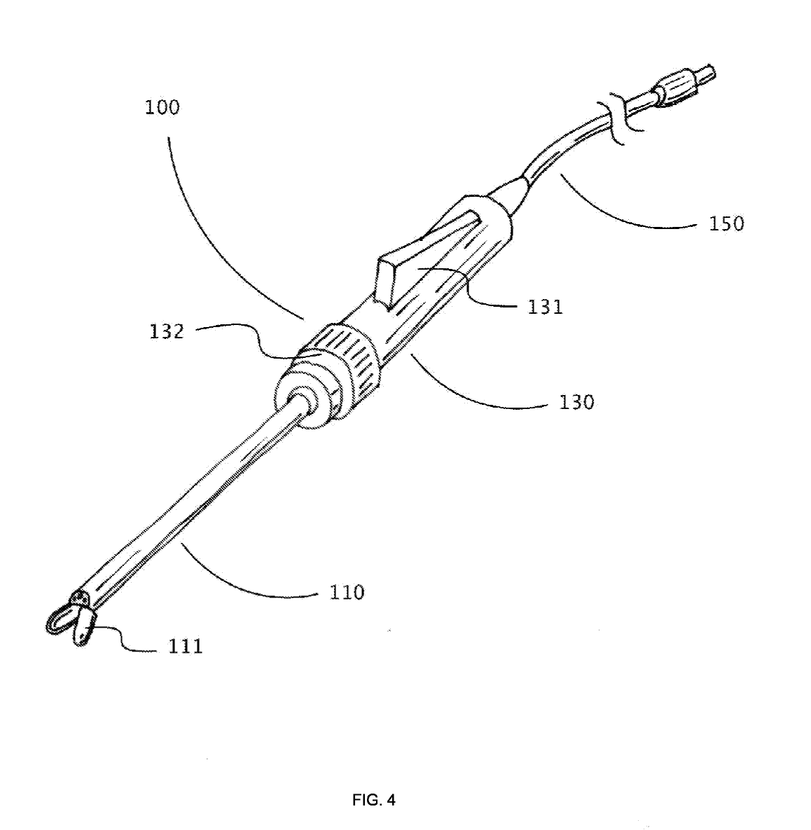

FIG. 4 provides an elevational view of an embodiment. In this embodiment, the scope (100) includes a distal elongate member (110), a handle (130), and a cable (150) which connects to a console 300 (not shown). In FIG. 4, the scope (100) is depicted with the tissue collection actuator (131) in a first position and the tissue collection component (111) open, and the retroflex actuator (132) in a first position and the elongate member (110) straight with the tissue collection component (111) directed away from the handle (130).

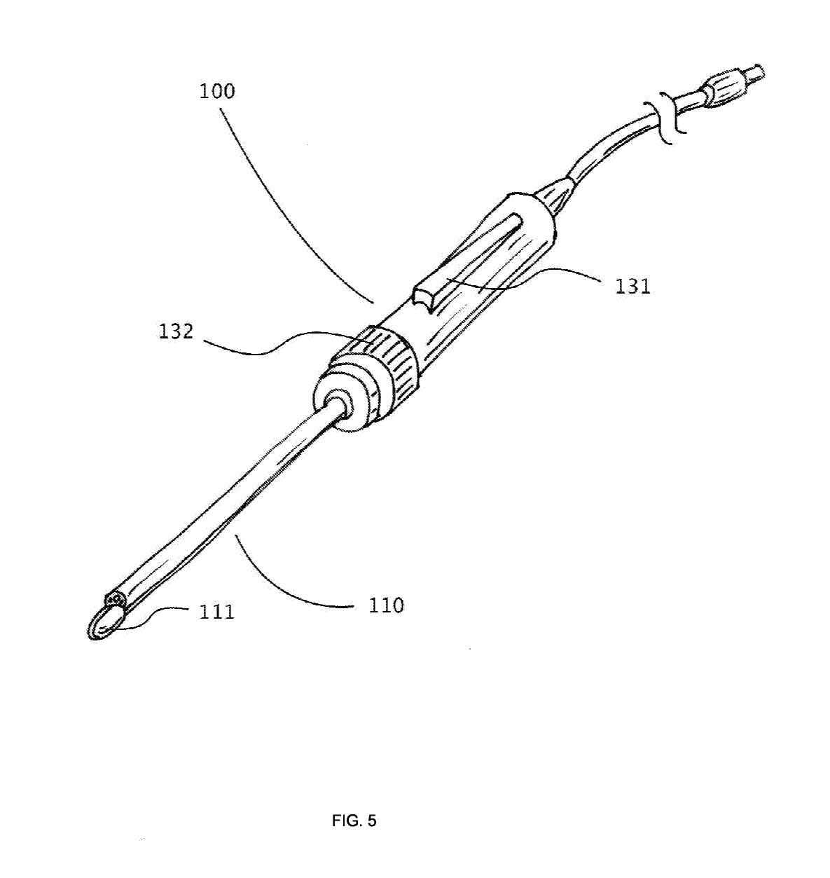

FIG. 5 provides an elevational view of the same embodiment of the scope (100) with the tissue collection actuator (131) moved into a second position and the tissue collection component (111) closed. The retroflex actuator (132) remains in a first position with the elongate member (110) straight.

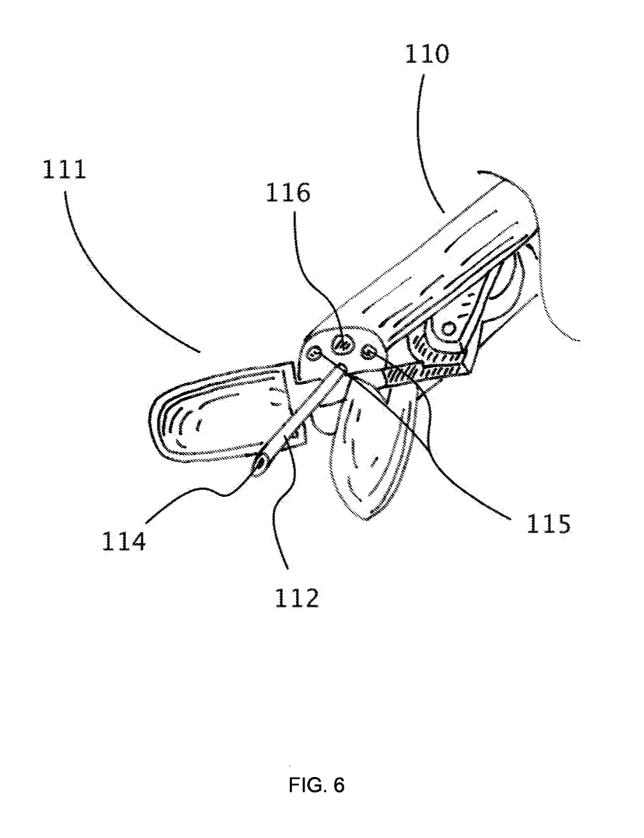

FIG. 6 is a close-up elevational view of one embodiment of the tissue collection component (111) located at the distal end of the elongate member (110) in the open position. In this embodiment, when the tissue collection component (111) is open, a fluid transmission component (112) is visible. Adjacent to the tissue collection component (111) are at least one energy emitter (115) and at least one energy detector (116). When in operation with the tissue collection component, the energy emitter (115) emits energy that passes through the pleural effusion fluid (15), reflects off of the pleural surfaces, and is received by the energy detector (116). Because the pleural effusion fluid (15) is often turbid and ranges in color from translucent yellow and brown to nearly opaque red and even white, the energy emitter (115) and energy detector (116) are located close to the tip of the tissue collection component (111), within the range of 0 to 40 mm, preferable within the range of 1 to 20 mm and ideally within the range of 1-5 mm. The energy that can be emitted and detected could be broad-band light energy from the visible spectrum, a narrow-band of light energy from the visible spectrum, infra-red light energy, ultra-violet light energy, polarized light energy, coherent light energy, radio waves, x-rays, microwave energy, sound energy, ultrasound energy, and the like.

In some embodiments a single energy emitter (115) and single energy detector (116) are desirable. As an example, when the energy emitter (115) is designed to emit photons of light energy in the entire visible spectrum and the energy detector (116) is designed to detect photons from the entire visible spectrum of light energy or a narrow band of energy from the visible spectrum or a single wavelength of light.

Example optical energy emitters include single, multiple fiber optic fiber cables or light emitting diodes. The preferred optical energy emitter (115) is a light emitting diode (“LED”). Compact LEDs approximately 1.0×1.0 mm in size or smaller and emitting light intensity levels ranging from approximately 50 to 100 millicandela or more are used in some embodiments. Example optical energy detectors include charge coupled devices (CCDs) or Complementary Metal Oxide Semiconductor (CMOS) focal plane array chips. In one embodiment, the optical energy detector is integrated into a color camera module. The module preferably, but is not required to, includes at least one optical filter, for example a Bayer pattern color filter array is used for color images. Other optical filters or polarizing filters could be incorporated into the camera module as well. The module also preferably includes at least one optical lens. The lens system has an F number preferably ranging from 2.7 to 6.0 or greater. The lens depth of focus ranges from preferably 3 mm to 50 mm or less. The camera module enables real-time acquisition and viewing of pleural tissue of static images or moving images (i.e. video) via a signal connection to an image viewing module.

In some embodiments the signal connection from the camera module to the imaging viewing module is accomplished through a wired connection which includes the following functions: supplying power from the imaging module to the camera module and LEDs, and supplying camera image signals to the imaging module. In alternative embodiments the scope (100) includes a power source such as a battery as well as a wireless link to connect control and signals for both the energy emitter and energy detector with the imaging module. Examples of wireless links include Bluetooth® transceiver modules that are capable of high speed communications which are compatible with transmitting both static and dynamic images (e.g. video). In some embodiments, the wireless link also controls when the energy emitter is emitting energy or other functions of the device. Advantages of a wireless embodiment include ease of use, improved electrical safety, fewer cables and mechanical connections, and lower cost.

In another embodiment, the energy emitter (115) and energy detector (116) are a single entity that is capable of both emitting energy and detecting energy. As an example, the energy may be ultrasound energy and the combined energy emitter/detector is an ultrasound transducer operating in the pulse-echo mode of imaging.

In some embodiments, a single energy emitter (115) could be used with a plurality of energy detectors (116), each detector designed to receive different types of energy. In one embodiment, a single energy emitter (115) could emit light energy in the visible spectrum and each detector (116) could detect light polarized to 0 and 90 degrees. In one embodiment, the energy emitter (115) could emit light energy in the visible spectrum and each energy detector (116) could be designed to receive light from a specific wavelength or band of wavelengths. In some embodiments, the energy emitter (115) is used in conjunction with one or more energy filters that alter the band of wavelengths emitted from the scope (100). For instance, one or more energy filters could be used to alter the wavelengths that could be detected by the energy detectors (116).

Alternatively, one or more energy emitter (115) emitting different types of energy could be used with one or more energy detectors (116).

In some embodiments, the fluid transmission component (112) has at least one lumen (114) that passes through the elongate member (110), the handle (130) and the cable (150) to connect to a fluid connector (not shown) in the cable (150). The at least one lumen (114) can be used to aspirate pleural effusion (15) fluid from the pleural space (10) for analysis. In some embodiments, the at least one lumen (114) can also be used to pass fluid through the fluid transmission component (112) and into the pleural space (10) thereby diluting the pleural effusion (15) fluid. Fluid may be delivered once, repeated intermittently, or continuously. The fluid may be optically clear or transparent such as solutions of saline, dextrose, or the like. The fluid may be used to dilute the pleural effusion fluid (15) near the tip of the elongate member (110) thereby decreasing turbidity and improving translucency. This may improve energy transmission between the energy emitter (115), the parietal (12) and visceral (13) pleural surfaces and the energy detector (116) thereby improving visualization of the parietal (12) and visceral (13) pleural surfaces. In some embodiments, the fluid may contain an anesthetic to anesthetize the parietal (12) and visceral pleural surfaces prior to manipulation or biopsy. If, for example, the fluid includes lidocaine or the like, the fluid will provide an anesthetic effect and also dilute the pleural effusion fluid (15) to improve visualization. Additionally, the fluid can be added to displace lung tissue thereby creating space near the pleural surface and further enhance visualization and the ability to position the tissue collection component (111) near the desired tissue collection site. In some embodiments, the fluid transmission component (112) may be used to remove fluid from the pleural space. Some embodiments have at least one lumen for fluid delivery through the needle (112) as well as at least one lumen (not shown) for fluid delivery (e.g. optically clear fluid) and/or aspiration of pleural fluid. In some embodiments, the fluid includes a viscosity modification agent.

In one embodiment, the tissue collection component (111) is a cutting forceps, but in other embodiments the tissue collection component (111) could be an alligator forceps, a rat tooth forceps, a punch biopsy device, an aspiration needle, a cutting needle, a hook, a tissue abrader, a cryo probe, or the like.

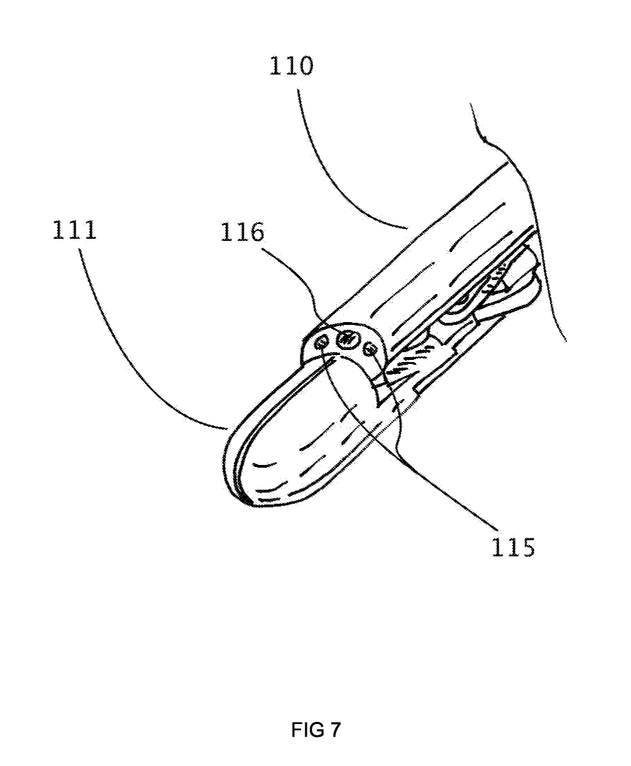

In one embodiment, the fluid transmission component (112) is a needle that is positioned to extend beyond the distal tip of the tissue collection component (111) when opened as shown in FIG. 6 and be enclosed within the tissue collection component (111) when closed as shown in FIG. 7. The needle fluid transmission component (111) can be inserted into the parietal pleura and used to inject fluid into the parietal pleura. In some applications, the fluid may include an anesthetic such as lidocaine or the like. The needle may be integrated with the device and locked in a fixed in location relative to the tissue collection component (111) or its position can be adjustable or it can be removable. The elongated member (110), energy emitter (115), and energy detector (116) are visible in this view.

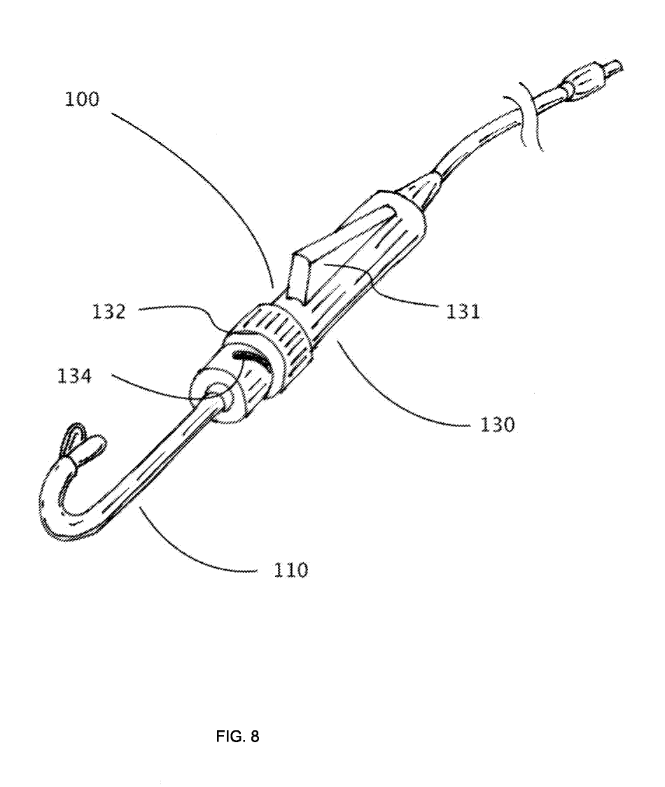

FIG. 8 provides an elevational view of one embodiment of the scope (100) with the tissue collection actuator (131) in a first position and the tissue collection component (111) open. The retroflex actuator (132) is moved into a second position that causes the elongate member (110) to flex. In one embodiment, the retroflex actuator (132) is designed to engage threads (134) in the handle (130) and can be moved from a first position to a second position by rotation around the long axis of the handle. Movement of the retroflex actuator (132) produces flexion of the elongate member (110) from a straight position to some desired second degree of flexion. In some embodiments, the retroflex actuator (132) can be moved from the second position to a third position to cause a third degree of flexion of the elongate member 110. This configuration allows the degree of flexion in the elongate member (110) to be adjusted between 0 and 270 degrees and remain fixed at any desired degree until the actuator is rotated to a new position. In one embodiment, the degree of flexion of the elongate member to be adjustable in fixed angles which may include user interface indications such as marks, letters, or numbers located on or near the on the retroflex activator (132). In one embodiment, audible snaps or clicks to provide indication to the user that the flexion angle of the elongate member has been changed or has been set to a particular angle.

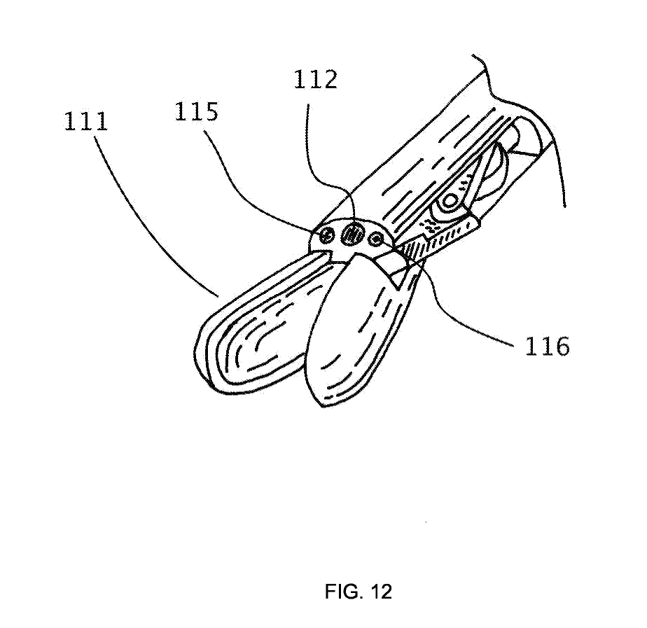

The size and shape of the scope (100) is optimized for diagnostic pleuroscopy. The distal elongate member (110) has a rigid section, a flexible section and a distal section (shown in FIG. 12). The length of the rigid section is optimized by reducing its length compared to reusable pleuroscopes in the state of the art. A shorter scope length gives the user improved positional control of the scope distal end and improves targeting. Embodiments of the scope have a rigid length which ranges in length from 50-100 cm, 100-150 cm, 150-200 cm and is less than 150, 200, or 250 cm in length. A preferred embodiment length is 100-150 cm. The diameter of the distal elongate member (110) is optimized to reduce the size/area of the skin incision. A smaller incision size improves wound healing, reduces pneumothorax risk and minimizes infection risk. Embodiments of the scope have a distal elongate member (110) diameter which ranges from 2-3 mm, 3-4 mm, 4-5 mm, 5-6 mm and is less than 2, 4, preferably 5, 6, or 7 mm in diameter. In a preferred embodiment, the scope has 5 mm diameter or less. For example, a 5 mm diameter requires a skin incision area of approximately 19.6 mm2. (This is less than half incision area required by the current state of the art scope.)

Integrating the tissue collection component (111) with the energy emitter (115) and the energy detector (116) allows increasing the size of the tissue collection component relative to the current state of the art. The current state of the art uses biopsy forceps approximately 2 mm in diameter or smaller (cross sectional area approximately equal to 12 mm2). Embodiments of the scope (100) have a tissue collection component of 20, 30, 40 or 50 mm2 or greater in cross sectional area. A preferred embodiment has a tissue collection component between 25 and 50 mm2 in cross sectional area. The increased size of the tissue collection component increases diagnostic yield and reduces the number of tissue samples passes needed for a particular procedure relative the current state of the art. Fewer sampling passes preserves the cutting edge of the tissue sampling component. Fewer sampling passes reduces the number of insertions into and removals from the pleural space, reducing the risk of contamination or infection.

Because of the integrated design of the disclosed system, in some embodiments it can achieve high ratios of tissue sampling component cross sectional area to access cross section area.

The mechanical properties and architecture of the scope embodiments take advantage of the physiological/anatomical properties which result in a significant percentage of exudative pleural effusion conditions that adversely affect the architecture and fluid drainage characteristics of the lymphatic stoma and vessels located on the costal pleural. Targeting sampling of abnormal parietal pleura tissue in these areas gives a high yield for disease diagnosis. The scope is optimized (e.g. larger tissue sample size, smaller incision relative to sample size, improved user controls of scope distal end, enables imaging in the presence of turbid pleural fluid and is single-use) to sample pleural tissue in these targeted areas to increase diagnostic yield, improve usability, decrease procedure time, decrease infection rates and save cost over the current state of the art.

In some embodiments, the scope (100) includes a joint to facilitate a tight bend within the pleural space. State of the art endoscopes have a limited radius of curvature to preserve passage of implements like the biopsy forceps or sampling needles through the working channel lumen. Similarly, tight curvatures can cause the working channel lumen to bind on the passed devices, limiting their ability to actuate or pass fluid. In contrast, because the joint is designed in, the device can preserve the ability to actuate the tissue collection component (111) when the elongated member (110) is placed into a tight curvature. Similarly, the actuation state of the tissue collection component (111) can be preserved during the bending of the elongated member (110) of the scope.

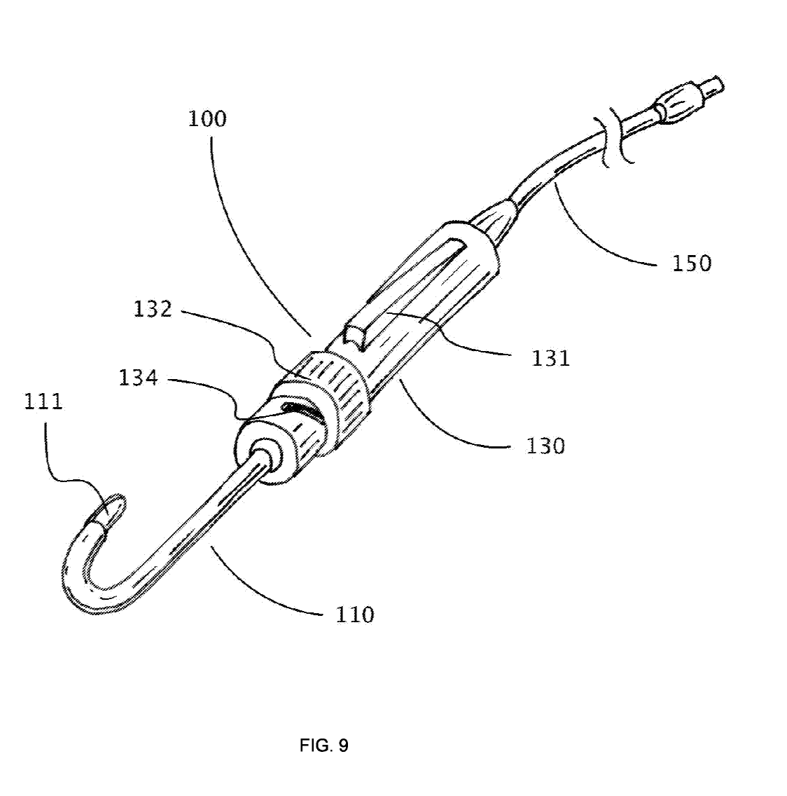

FIG. 9 provides an elevational view of an embodiment of the scope (100) with the retroflex actuator (132) in a second position with the elongate member (110) flexed and the tissue collection actuator (131) in a second position causing the tissue collection component (111) to be closed. The handle (130), threads (134), and cable (150) are also evident in this figure.

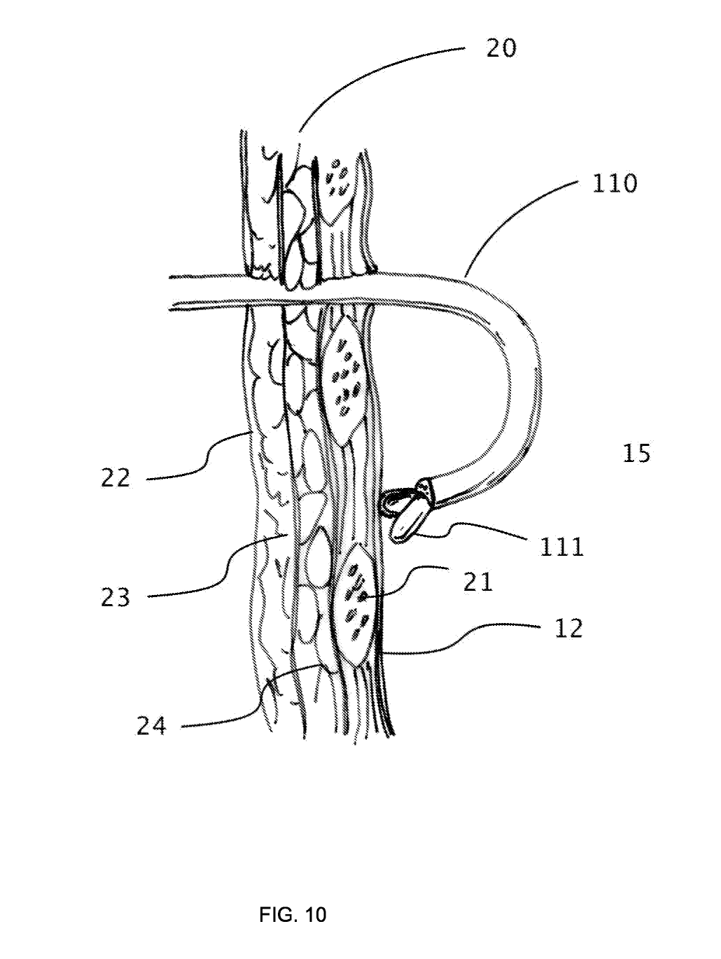

FIG. 10 is a cross sectional view of the chest wall (20) with the elongate member (110) of the scope (100) passed through the skin (22), fat (23), and muscle (24) along the superior margin of a rib (21) into a pleural effusion (15). The elongate member (110) is in a flexed position with the tissue collection component (111) opened and in position to collect a tissue specimen from the parietal pleura (12). Prior to collection of a tissue specimen, anesthetic can be passed through a lumen (114) of the fluid transmission component (112) and instilled into the fluid of the pleural effusion (15) near the planned tissue collection site of the parietal pleura (12). The local instillation of anesthetic into the pleural effusion (15) fluid serves to at least partially anesthetize the parietal pleura (12). In one embodiment at least one needle fluid transmission component (112) of the scope can also or alternatively be inserted through the parietal pleura (12) and anesthetic can be injected directly into the chest wall (20) to anesthetize the parietal pleura (12). The tissue collecting component (111) can then be brought into contact with the parietal pleura at the desired tissue collection site and the tissue collection actuator (131) is activated closing the tissue collecting component (111) and entrapping a specimen of the parietal pleura (12). With the tissue specimen contained in the tissue collecting component (111), the retroflex actuator (132) can then returned to the first position to straighten the elongate member (110) and the scope (100) can be removed from the chest wall (20). The tissue collecting component (111) can then opened and the tissue specimen can be removed.

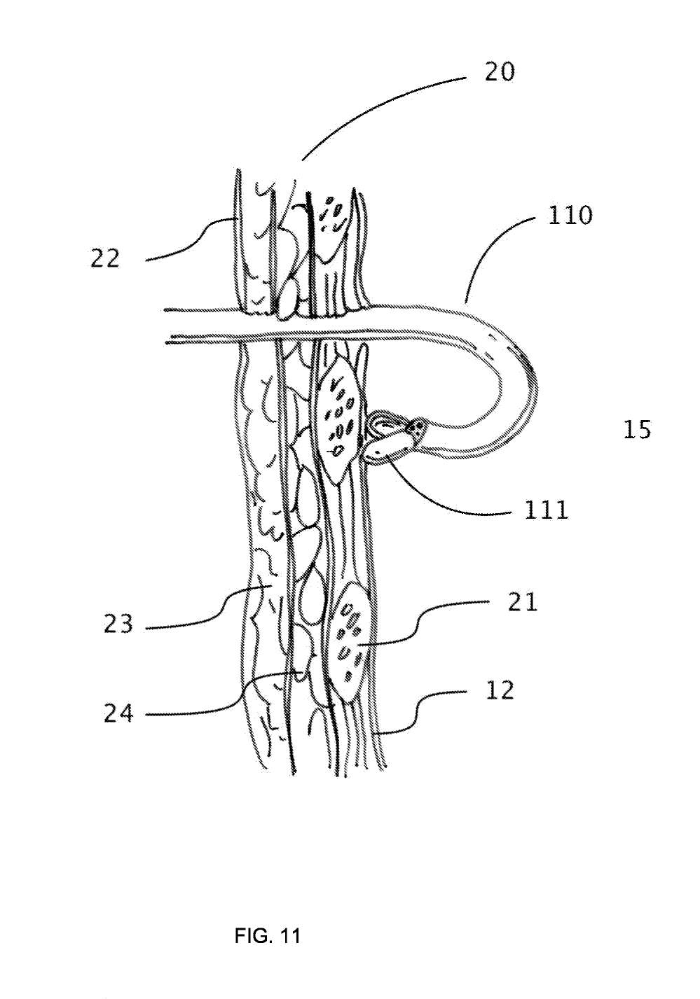

FIG. 11 shows that the scope (100) can be reinserted into the pleural effusion (15) through the same incision in the chest wall (20) and the retroflex actuator (132) can be moved to a different position to cause a different desired degree of flexion of the elongate member (110) to bring the tissue collection component into close proximity to a different portion of the parietal pleural (12).

FIG. 12 shows an embodiment of the distal end of the elongate member (110) with the tissue collection component (111) in the open position. Adjacent to the tissue collection component (111) is a fluid transmission component (112), an energy emitter (115) and an energy detector (116). This configuration has the advantage of positioning the fluid transmission component immediate adjacent to the energy emitter (115) and detector (116) thereby enhancing pleural effusion (15) fluid dilution that occurs with the delivery of fluid and improved visualization.

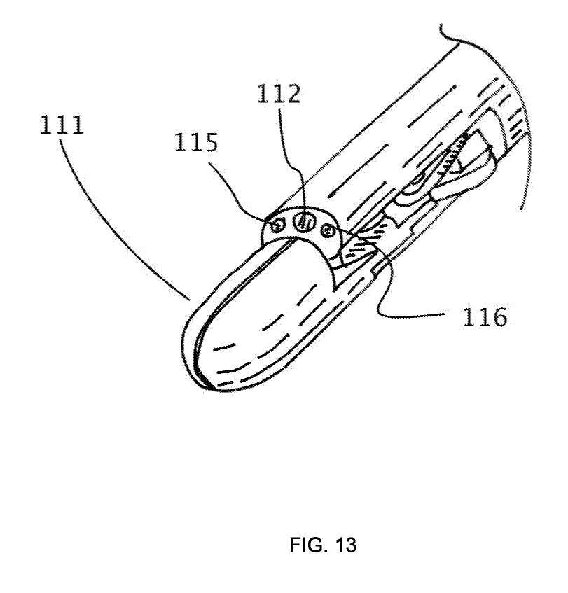

FIG. 13 shows an elevational view of the same embodiment with the tissue collection component (111) in the closed position. The fluid transmission component (112), energy emitter (115), and energy detector (116) are visible.

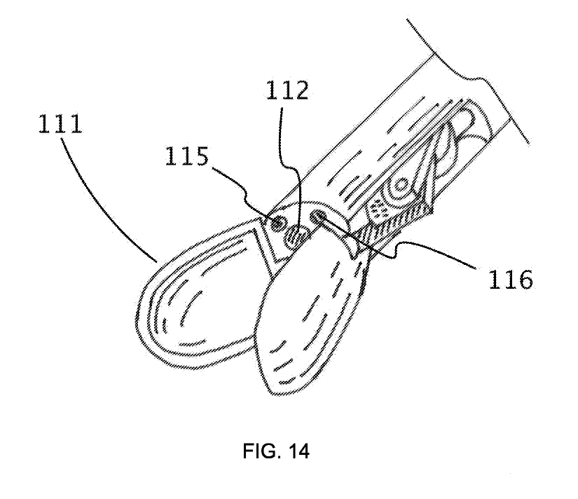

FIG. 14 shows an alternative embodiment of the distal end of the elongate member (110) with the tissue collection component (111) in the open position. In this embodiment a fluid transmission component (112), an energy emitter (115) and an energy detector (116) are confined within the tissue collection component (111). Emission of energy to enable detection and/or visualization of the pleural surfaces is performed while the tissue collection component (111) is in the open position. Fluid can be delivered through a fluid transmission component (112) to dilute the pleural effusion fluid contained within the space of the open tissue collection component (111). In this configuration, the tissue collection component (111) serves to partially contain the diluted pleural effusion fluid and enhance energy transmission, detection and/or visualization.

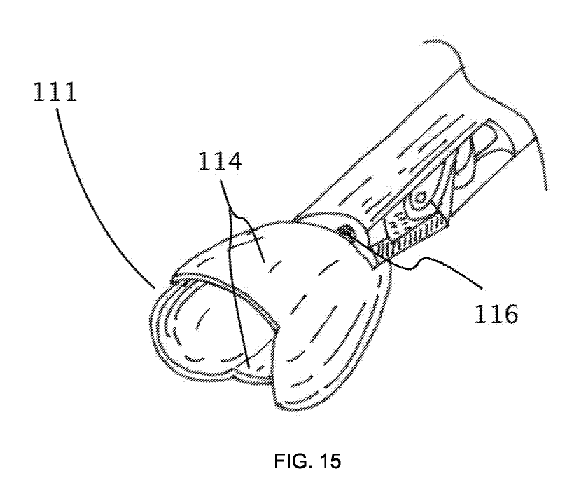

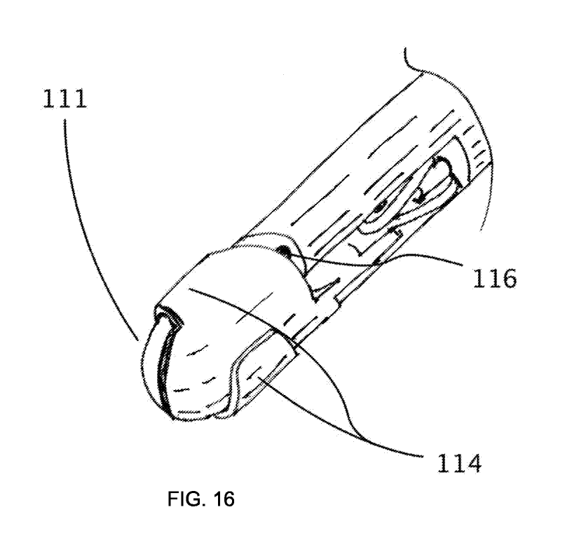

FIG. 15 shows an embodiment in which flexible extensions (114) have been added to the tissue collection component (111) to enhance the containment of the diluted pleural effusion fluid and further enhance energy transmission, detection and/or visualization. These extensions (114) are ideally constructed of a spring-like material that allows them to be pushed apart when the tissue collection component (111) is open and recoil, as shown in FIG. 16, to wrap around the closed tissue collection component (111). Alternatively these extensions could be rigid, hinged, or a flexible membrane enclosing the space within the tissue collection component (111) when in the open position. The turbid fluid within this volume may be displaced by an optically clear fluid to improve visualization of the target tissue. An alternative embodiment uses a hood over the distal end of the scope and displacement with an optically clear fluid to isolate the imaging component from the turbid fluid and optimize imaging.

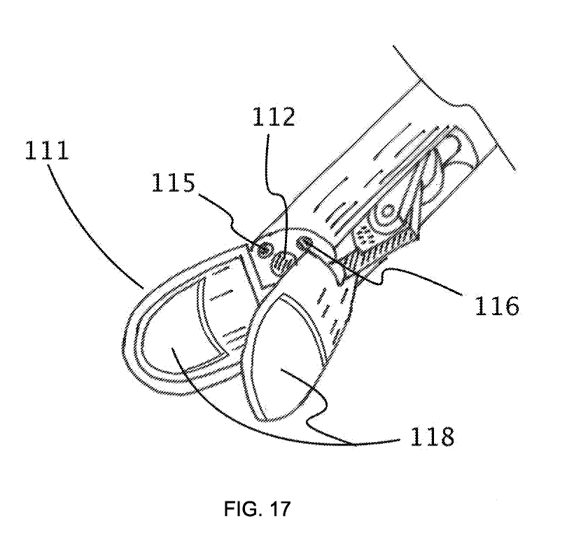

FIG. 17 shows an embodiment of the distal end of the elongate member (110) with the tissue collection component (111) in the closed position. In this embodiment, a fluid transmission component (112), an energy emitter (115) and an energy detector (116) are confined within the tissue collection component (111). The portion of the tissue collection component (111) between the energy emitter (115) and the pleural surfaces forms an energy transmission window 118 that is composed of a material that is transparent or translucent to the energy emitted by the energy emitter (115). In the case of light energy for example, the tissue collection component could be composed of glass, quartz, polycarbonate, acrylic, and the like. Alternatively the energy transmission window 118 could be a cut-out with no material.

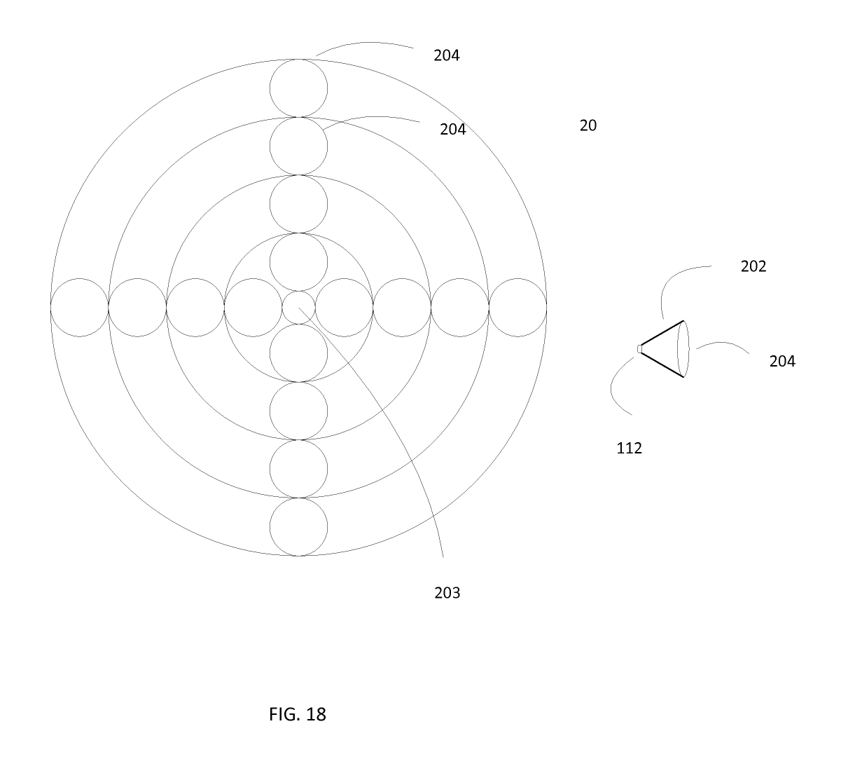

FIG. 18 is a view of the chest wall (20) imaging plane search pattern as seen from the inside of the patient's body. In an embodiment that uses optical imaging, the camera module imaging plane (204) is determined by the focal length and F number of the camera module optics as well as the distance from the lens to the target image. The angular aperture of camera module lens (202) is the apparent angle of the lens aperture as seen from imaging device (i.e. CCD or CMOS chip). The insertion area of the elongate member (203) into the chest wall (20) defines the center of the parietal pleural area that is intended for imaging and sampling of abnormal tissue within the area. A circular search pattern is determined by concentric rings which are approximately equal in distance the camera module imaging plane diameter. In one embodiment, markings are provided on the elongate member (110) to allow the user to adjust the depth of implantation in the patient's chest. These marking also allow the user to return to the approximate depth of elongate member (110) implantation after removal and re-insertion which occurs, for example when a biopsy sample, is removed from the patient.

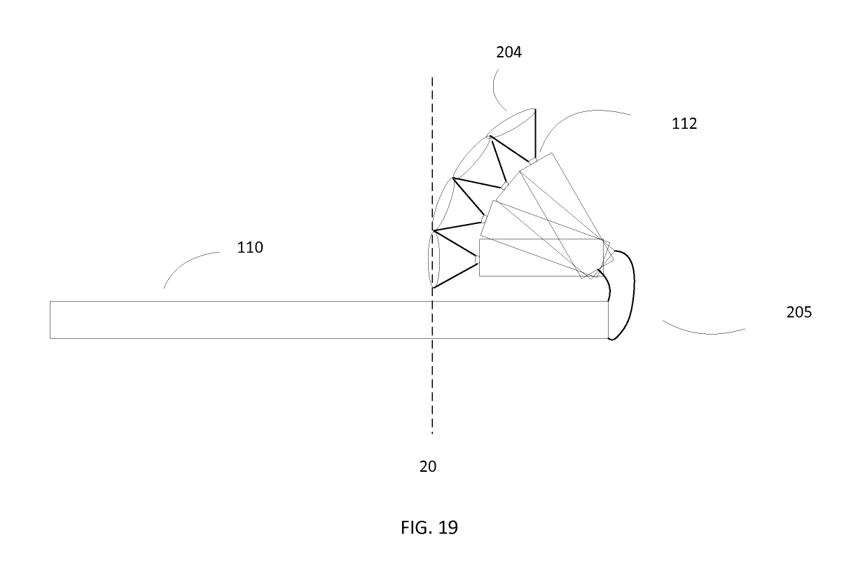

FIG. 19 is a view of the scope with the elongate member (110) flexed at angles which define a circular search pattern on the parietal pleura located on the inside of the chest wall (20). The location of the flexure joint (205) is shown on the elongated member (110). The angulation of the flexure joint determines the distance of the imaging plane from the elongate body.

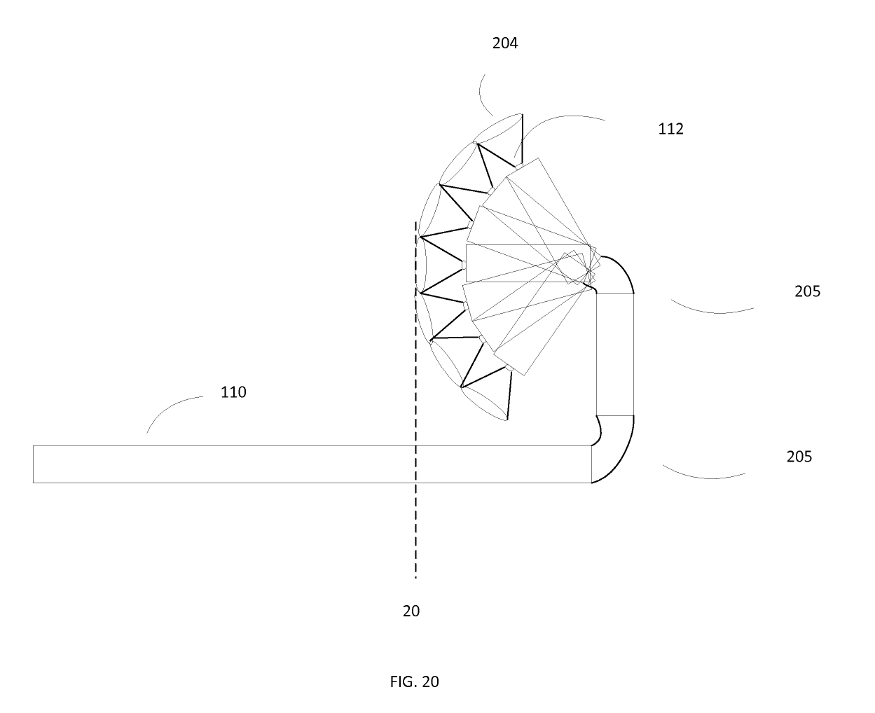

FIG. 20 is a view of an embodiment which has a plurality of locations whereby the elongate member flexes which enables an expanded circular search pattern with a single location of implantation,

The scope may be used with a flexible trocar. The flexible trocar reduces stimulation to the intercostal nerves and allows for the incision site to stay open between scope removals and re-insertions (e.g., after obtaining a biopsy sample). In some embodiments of the flexible trocar includes features for reducing or preventing leakage of pleural fluid between removal and re-insertion of the scope.

A preferred embodiment is a single-use (or disposable) scope. Reusable devices greatly increase the risk of infection and cross contamination (relative to single-use devices) due to the potential for a poorly sterilized device to be used with a patient's procedure. Single-use devices also eliminate the cleaning and sterilization preparation requirements of reusable scopes. Technology improvements and significant cost reductions in energy emitting (for example LEDs) and energy detecting technologies (for example CMOS imaging chips) no longer make fabrication of a single-use scope cost prohibitive. Additionally, single or limited-use tissue collection component (for example biopsy forceps) use sharp cutting edges which make acquisition of tissue samples easier for the user and less traumatic for the patient. Reusable biopsy forceps have duller edges which makes tissue sample acquisition more difficult.

Although single-use medical devices have the aforementioned advantages over reusable medical devices they are often reprocessed and reused by the end users in an effort to save cost. Since most of the these devices were never intended for cleaning, re-sterilization and reuse the unintended reuse degrades device performance and can significantly increase patient risk. In some embodiments, the scope includes anti-reuse devices. In one embodiment, usage of a unique wireless Bluetooth ID number for a particular scope starts a countdown clock that allows for usage of the scope for a set time period after starting a procedure, for example 2 hours. In another embodiment, an integrated circuit chip in the scope electrical connector uses a similar method as the unique Bluetooth ID. In another embodiment, a fuse is incorporated in the scope electrical connector. This fuse is blown (or electrically opened) upon first usage of the device. When the fuse is blown a similar countdown clock is started.

In some embodiments, optical signal processing is used to improve imaging quality through turbid pleural fluid. In some embodiments which use light energy, the scope includes optical signal processing to minimize the optical light backscatter generated by the turbid pleural fluid. In one embodiment, a single polarization filter is used which can be rotated by either rotating the scope handle or adjusting the filter angle to reduce scatter due to ambient light. In another embodiment, Differential Orthogonal Polarization Imaging (DOPI) is used. DOPI subtracts two imaging frames taken from the same or similar viewing location. Each imaging frame is acquired with polarization at 90 degree rotational angles relative to other imaging frame. This method suppresses imaging background scatter that is common to both images (i.e. common-mode) because both light scattering will be of similar intensity in both images due to the homogeneity of scattering in turbid fluids for example pleural fluids. The target image of interest when acquired in this manner will have different optical intensities when imaged through a polarizing filter oriented at two rotational angles oriented 90 degrees to each other. Since the target image characteristics will be dissimilar in the two image planes subtracting the two images will yield desirable target image information. In one embodiment, imaging signal processing is accomplished with two imaging components (for example two camera modules) each including a polarization filter oriented at 90 degrees relative to the second polarization filter. Alternatively, DOPI could be accomplished with one imaging component and one polarization filter which changes orientation by 90 degrees between each imaging frame. In one embodiment the polarization filter is electrically activated and has at least two states of operation whereby at least one state of operation includes optical polarization oriented at 90 degrees relative to another state of operation.

In one embodiment, an imaging processing algorithm is used to suppress background light scattering. For example, the scattered light intensity is homogeneous over a short spatial distance whereby the target image will have larger changes in intensity over the same spatial distance. An algorithm which subtracts or rejects light intensity that is constant over a short distance decreases light scatter due to the homogeneous turbid fluid and increases the contrast of the target image. Image processing can improve the maximum viewing distance.

In some embodiments, the integration enables the use of a smaller access. In some embodiments, the integration enables retrieval of larger tissue samples. In some embodiments, the integration reduces the number of components that must be manipulated by the surgical staff during the procedure. In some embodiments, the integration reduces the complexity of the device. In some embodiments, the integration reduces the risk of incomplete sterilization. In some embodiments, the working length of the device is short compared with conventional devices, allowing for improved control.

In some embodiments, the device includes one or more lumens between the distal portion and proximal portions of the device. In some embodiments, at least one such lumen is connected to a needle on the distal portion of the device. In some embodiments, the needle may be used to inject analgesic into the pleural fluid near the biopsy site. In some methods, the needle is used to inject analgesic into the tissue at or near the biopsy site. In some embodiments the tip of the needle is distal to the open tissue collection component while the tip of the needle is proximal to the closed tissue collection component.

In some embodiments, at least one such lumen may be used to sample pleural fluid. In some embodiments, at least one such lumen may be used to introduce clear fluid into the pleural space. In some embodiments the fluid is optically clear. In some embodiments, the pleural fluid is clear to another form of imaging such as IR, UV, or ultrasound. In some embodiments the clear fluid is saline. In some embodiments the clear fluid includes a thickening agent.

In some embodiments, the one or more of the lumens is located within the tissue collection component. In some embodiments, the one or more lumens is located outside of the tissue collection component.

In some embodiments, one or more energy emitters are located within the tissues collection component. In some embodiments, one or more energy detectors are located within the tissue collection component.

In some embodiments, one or more energy emitters are located outside the tissues collection component. In some embodiments, one or more energy detectors are located outside the tissue collection component.

In some embodiments, one or more joints allow localized bending of the endoscope. In some embodiments, the endoscope is capable of greater than 150 degree bends. In some embodiments, the endoscope is capable of greater than 180 degree bends. In some embodiments, a plurality of joints allow bending in more than one plane.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A system for evaluating the pleural space comprising:
a device with a distal portion and a proximal portion, wherein the distal portion configured be introduced percutaneously into a pleural space;
a tissue collection component integrated in the distal portion of the device;
an energy detector located on the distal portion of the device; and
a joint have a range of motion and capable of being actuated from the proximal portion of the device, wherein actuation produces a bend of at least 120 degrees in the distal portion of the device, and
the tissue collection component functions over the range of motion of the joint.

2. The system of claim 1, further comprising:
a lumen connecting the distal portion and the proximal portion of the device, wherein the lumen accommodates both injection and extraction of fluid and
an energy emitter, located on the distal portion of the device.

3. The system of claim 2, further comprising:
a hollow needle located on the distal portion of the device, the needle being connected to the lumen, wherein the needle is configured to inject material.

4. The system of claim 1, wherein
the joint allows the tissue sampling device to be brought into contact with the parietal pleura anywhere over a range of 1 to 5 cm from where the device is inserted through the pleura.

5. The system of claim 1, wherein the distal portion of the device can pass through a 7 mm diameter opening.

6. The system of claim 1, wherein the energy detector is a plurality of energy detectors.

7. The system of claim 6, wherein the plurality of energy detectors are filtered spatially orthogonal so as to detect polarized electromagnetic radiation.

8. The system of claim 1, wherein the tissue collection component further comprises a cutter that covers the energy detector when the cutter is closed and wherein a portion of the cutter permits transmission of energy of the type detected by the energy detector.

9. The system of claim 2, further comprising:
a hood attached to the distal portion of the device, wherein the hood limits mixing of fluid near the energy detector, allowing injection of optically clear fluid near the energy detector to displace the pleural fluid.

10. A pleural biopsy device, comprising:
a system for imaging a parietal pleura and
a tissue sampling component capable of sampling and retaining a portion of the parietal pleura 20 to 50 square mm in area over a range of 1 cm to 5 cm away from where the device passes percutaneously through the parietal pleura.

11. The device of claim 10, wherein the device is used without inducing pneumothorax in a patient whose parietal pleura is being sampled.

12. The device of claim 11, wherein the imaging system provides optical images to a user.

13. The device of claim 11, wherein a ratio of cross sectional area of the sampled parietal pleura to a cross section area of the distal portion of the device is from 0.69 to 2.55.

14. A method of selectively sampling a pleura of a patient with pleural effusion without inducing pneumothorax, the method comprising:
introducing a device percutaneously into a plural space of a patient below the level of the pleural effusion and into the pleural effusion fluid;
evaluating the pleura using an energy emitter and energy detector integrated in the device, and
sampling the pleura in the region around a point of percutaneous access using a biopsy tool integrated into the device, wherein pneumothorax is not induced in the patient.

15. The method of claim 14 wherein the method further comprises introducing a fluid through the device into the pleural space.

16. The method of claim 15, wherein the energy detector is a color camera.

17. The method of claim 14, wherein the method further comprises:
introducing an analgesic through the device.

18. The method of claim 14, wherein information from the energy detector is provided wirelessly to a receiver external to the pleural space.

19. The method of claim 14, wherein an access used to introduce the device into the pleural space is smaller than 7 mm in diameter.

20. The method of claim 14, wherein the patient does not receive one or more of the following: conscious sedation and general anesthesia.
